(19) United States
(12) Patent Application Publication
(10) Pub. No.: US 2018/0110895 A1
(43) Pub. Date: Apr. 26, 2018

(54) ABSORBENT ARTICLE WITH OXYGEN DELIVERY AND METHOD OF MANUFACTURE

(71) Applicant: Kimberly-Clark Worldwide, Inc., Neenah, WI (US)

(72) Inventors: John Gavin MacDonald, Decatur, GA (US); HyoungKun Park, Yongin-si (KR); Cai Shan Tan, Yongin-si (KR)

(21) Appl. No.: 15/571,206

(22) PCT Filed: Jun. 30, 2015
(86) PCT No.: PCT/US2015/038441

§ 371 Date: Nov. 1, 2017

(51) Int. Cl. A61L 15/18; A61F 13/535; A61L 15/42; A61L 15/60; A61L 15/20; A61L 15/26

(52) U.S. Cl. CPC A61L 15/18; A61F 13/535; A61L 15/425; A61F 2013/530832; A61L 15/20; A61L 15/26; A61L 15/60

(57) ABSTRACT

The present disclosure is directed to an absorbent article including a closed cell foam matrix for delivering oxygen. The absorbent article includes a liquid permeable inner surface for facing the wearer, an outer surface for facing away from the wearer, an absorbent assembly disposed therebetween, and a closed-cell foam matrix material applied to a portion of the absorbent article. The closed cell foam matrix contains a superabsorbent material and oxygen entrapped within the superabsorbent material. To produce the closed cell foam matrix for delivering oxygen, an alkali hydroxide catalyst and oxygen precursor are both added to an aqueous superabsorbent material. The aqueous solution is applied to a nonwoven material. The nonwoven material is then heated to produce oxygen by reacting the alkali hydroxide catalyst and the oxygen precursor thereby entrapping the oxygen in the formed closed cell foam matrix.

### BACKGROUND

Many known absorbent article configurations employ absorbent materials located between a liquid pervious topsheet and a vapor and liquid impermeable backsheet. Such backsheets are well suited to prevent the migration of liquid waste from the absorbent materials to the outer garments of a wearer. Unfortunately, the use of liquid and vapor impermeable backsheets can result in a relatively high degree of humidity within the diaper when in use. This may result in relatively high skin hydration levels and may lead to the onset of diaper rash.

In order to reduce the humidity level within diapers, breathable polymer films have been employed as outer covers for absorbent garments, such as disposable diapers The breathable films are typically constructed with micropores to provide desired levels of liquid impermeability and vapor permeability Other disposable diaper designs have been arranged to provide some level of breathability at the leg cuff regions of the diaper Still other disposable diaper designs have been arranged to provide humidity transfer regions in the form of breathable panels in otherwise vapor-impermeable backsheets or to employ perforated regions to help ventilate the garment.

In addition, due to the perception of leakage caused by excess water vapor passing through the backsheets and condensing on the clothing, there has been a trend to reduce the breathability of this outercover of absorbent articles. In fact, some diaper products have gone back to non-breathable films. Also, improvements in the fit of diapers further trap the micro-environment inside the diaper. These factors further increase the humidity and potentially reduce the oxygen concentration, since oxygen is being absorbed through the skin and also the skin microflora absorbs oxygen.

Thus, there is a need to provide an optimized product that delivers oxygen when insulted with moisture and absorb that moisture and humidity by the matrix that delivers oxygen on demand providing a feeling of freshness and odor control benefits while restoring the oxygen balance of the microenvironment.

### SUMMARY

The present disclosure is directed to an absorbent article including a closed cell foam matrix for delivering oxygen. The absorbent article includes a liquid permeable inner surface for facing the wearer, an outer surface for facing away from the wearer, an absorbent assembly disposed there between, and a closed-cell foam matrix material applied to a portion of the absorbent assembly.

The closed cell foam matrix contains a superabsorbent material and oxygen entrapped within the superabsorbent material. To produce the closed cell foam matrix for delivering oxygen, an alkali hydroxide catalyst and oxygen precursor are both added to an aqueous superabsorbent material. The aqueous solution is applied to a nonwoven material. The nonwoven material is then heated to produce oxygen by reacting the alkali hydroxide catalyst and the oxygen precursor thereby entrapping the oxygen in the formed closed cell foam matrix.

The superabsorbent material has at least 15 percent by mass monoethylenically unsaturated carboxylic, sulphonic or phosphoric acid or salts thereof, an acrylate or methacrylate ester that contains an alkoxysilane functionality, and a copolymerizable hydrophilic glycol containing ester monomer. Desirably, an aqueous solution of an oligomeric polyacrylic acid having a silanol cross-linker covalently bonded to the backbone chain of a polyacrylic acid is used for the superabsorbent material described herein.

Examples of alkali hydroxide catalyst that can be used include, but are not limited to, sodium hydroxide, lithium hydroxide, potassium hydroxide, magnesium hydroxide, calcium hydroxide, and combinations thereof. In desirable embodiments, the alkali hydroxide catalyst comprises sodium hydroxide. Suitably, the amount of the alkali hydroxide catalyst that is added may be between about 0.5 percent to about 3 percent by weight relative to the weight of the liquid superabsorbent polymer composition.

Examples of an oxygen precursor that can be used include, but are not limited to, hydrogen peroxide, ammonium peroxide, sodium peroxide, urea-peroxide, potassium percarbonate, and combinations thereof. In desirable embodiments, the oxygen precursor comprises hydrogen peroxide. Suitably, the amount of the oxygen precursor that is added may be between about 15 percent to about 25 percent by weight relative to the weight of the liquid superabsorbent polymer composition.

### DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 is a side view illustration of an embodiment of an absorbent article.

FIG. 2 is a top down view of an embodiment of an absorbent article with portions cut away for clarity.

FIG. 3 is an exploded cross-sectional view of an embodiment of an absorbent article.

FIG. 4 is an exploded cross-sectional view of another embodiment of an absorbent article.

FIG. 5 is an exploded cross-sectional view of another embodiment of an absorbent article.

FIG. 6 is an exploded cross-sectional view of another embodiment of an absorbent article.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DEFINITIONS

It is to be understood by one of ordinary skill in the art that the present discussion is a description of exemplary aspects of the present disclosure only, and is not intended as limiting the broader aspects of the present disclosure.

Within the context of this specification, each term or phrase below will include the following meaning or meanings.

“Bonded” refers to the joining, adhering, connecting, attaching, or the like, of two elements. Two elements will be considered to be bonded together when they are bonded directly to one another or indirectly to one another, such as when each is directly bonded to intermediate elements.

“Connected” refers to the joining, adhering, bonding, attaching, or the like, of two elements. Two elements will be considered to be connected together when they are connected directly to one another or indirectly to one another, such as when each is directly connected to intermediate elements.

“Cross direction” refers to the width of a fabric in a direction generally perpendicular to the direction in which it is produced, as opposed to “machine direction” that refers to the length of a fabric in the direction in which it is produced.

“Cross direction assembly” refers to a process in which disposable absorbent products are manufactured in an orientation in which the products are connected side-to-side, a process utilizing a cross direction assembly that entails products traveling through a converting machine parallel to the direction of arrow 49, as opposed to “machine direction assembly” in which the products are connected end-to-end or waist-to-waist.

“Disposable” refers to articles that are designed to be discarded after a limited use rather than being laundered or otherwise restored for reuse.

“Disposed,” “disposed on,” and variations thereof are intended to mean that one element can be integral with another element, or that one element can be a separate structure bonded to or placed with or placed near another element.

“Elastic,” “elasticized” and “elasticity” mean that property of a material or composite by virtue of which it tends to recover its original size and shape after removal of a force causing a deformation.

“Elastomeric” refers to a material or composite that can be elongated by at least 25 percent of its relaxed length and that will recover, upon release of the applied force, at least 10 percent of its elongation. It is generally preferred that the elastomeric material or composite be capable of being elongated by at least 100 percent, more preferably by at least 300 percent, of its relaxed length and recover, upon release of an applied force, at least 50 percent of its elongation.

“Fabrics” is used to refer to any woven, knitted and nonwoven fibrous webs.

“Film” refers to a thermoplastic film made using a film extrusion and/or forming process, such as a cast film or blown film extrusion process. The term includes apertured films, slit films, and other porous films that constitute liquid transfer films, as well as films that do not transfer liquid.

“Flexible” refers to materials that are compliant and that will readily conform to the general shape and contours of the wearer's body.

“Hydrophilic” describes fibers or the surfaces of fibers that are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 are designated “wettable” or hydrophilic, while fibers having contact angles greater than 90 are designated “nonwettable” or hydrophobic.

“Integral” or “integrally” is used to refer to various portions of a single unitary element rather than separate structures bonded to or placed with or placed near one another.

“Layer” when used in the singular can have the dual meaning of a single element or a plurality of elements.

“Liquid impermeable,” when used in describing a layer or multi-layer laminate, means that a liquid, such as urine, will not pass through the layer or laminate, under ordinary use conditions, in a direction generally perpendicular to the plane of the layer or laminate at the point of liquid contact. Liquid, or urine, can spread or be transported parallel to the plane of the liquid impermeable layer or laminate, but this is not considered to be within the meaning of “liquid impermeable” when used herein.

“Liquid permeable material” or “liquid water-permeable material” refers to a material present in one or more layers, such as a film, nonwoven fabric, or open-celled foam, which is porous, and which is water permeable due to the flow of water and other aqueous liquids through the pores. The pores in the film or foam, or spaces between fibers or filaments in a nonwoven web, are large enough and frequent enough to permit leakage and flow of liquid water through the material.

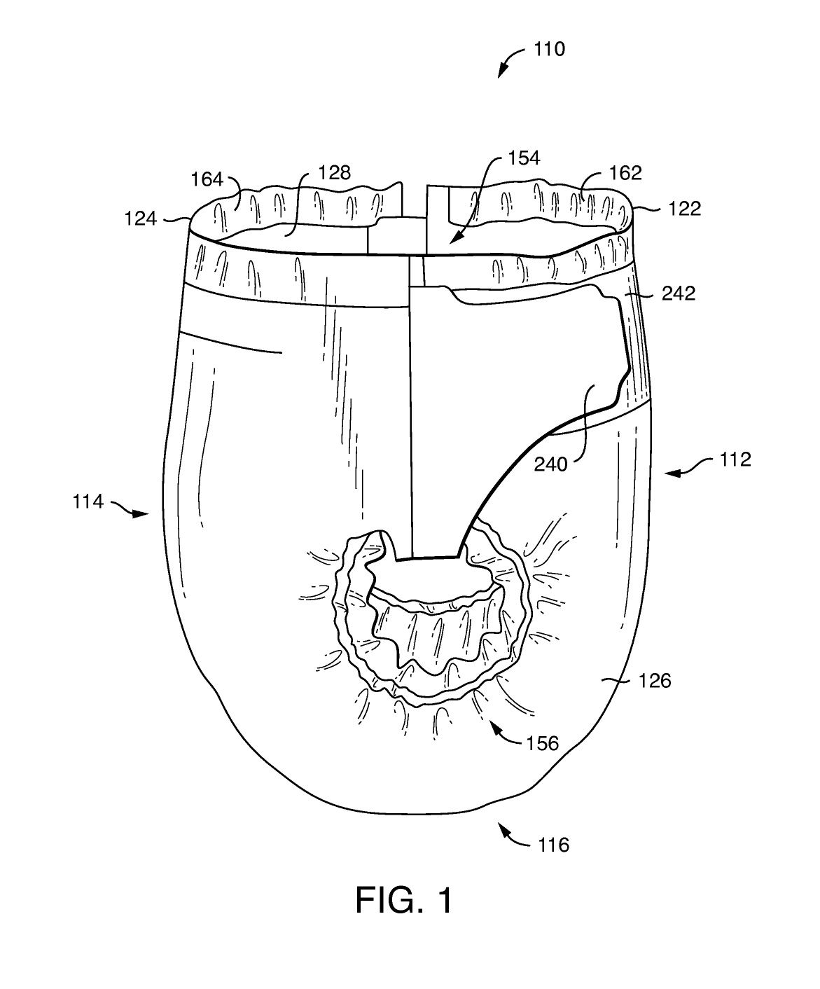

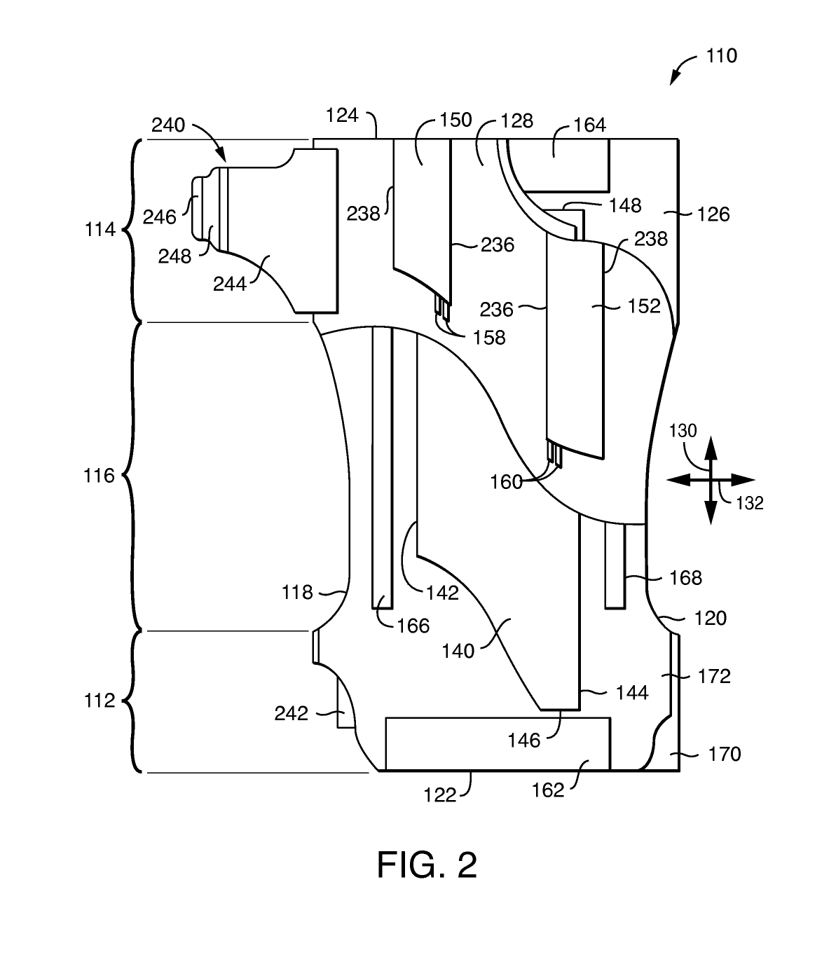

“Longitudinal” and “transverse” have their customary meaning, as indicated by the longitudinal and transverse axes depicted in FIGS. 1 and 2. The longitudinal axis lies in the plane of the article and is generally parallel to a vertical plane that bisects a standing wearer into left and right body halves when the article is worn. The transverse axis lies in the plane of the article generally perpendicular to the longitudinal axis. The article as illustrated is longer in the longitudinal direction than in the transverse direction.

“Machine direction” refers to the length of a fabric in the direction in which it is produced, as opposed to “cross direction” that refers to the width of a fabric in a direction generally perpendicular to the machine direction.

“Machine direction assembly” refers to a process in which disposable absorbent products are manufactured in an orientation in which the products are connected end-to-end or waist-to-waist, in the longitudinal direction shown by arrow 48 in FIGS. 1 and 2, a process utilizing a machine direction assembly entails products traveling through a converting machine parallel to the direction of arrow 48, as opposed to “cross direction assembly” in which the products are connected side-to-side.

“Meltblown fiber” means fibers formed by extruding a molten thermoplastic material through a plurality of fine, usually circular, die capillaries as molten threads or filaments into converging high velocity heated gas (e.g., air) streams that attenuate the filaments of molten thermoplastic material to reduce their diameter, which can be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers that can be continuous or discontinuous, are generally smaller than about 0.6 denier, and are generally self-bonding when deposited onto a collecting surface. Meltblown fibers used in the present disclosure are preferably substantially continuous in length.

“Member” when used in the singular can have the dual meaning of a single element or a plurality of elements.

“Nonwoven” and “nonwoven web” refer to materials and webs of material that are formed without the aid of a textile weaving or knitting process.

“Operatively joined,” in reference to the attachment of an elastic member to another element, means that the elastic member when attached to or connected to the element, or treated with heat or chemicals, by stretching, or the like, gives the element elastic properties; and with reference to the attachment of a non-elastic member to another element, means that the member and element can be attached in any suitable manner that permits or allows them to perform the intended or described function of the joinder. The joining, attaching, connecting or the like can be either directly, such as joining either member directly to an element, or can be indirectly by means of another member disposed between the first member and the first element.

“Permanently bonded” refers to the joining, adhering, connecting, attaching, or the like, of two elements of an absorbent garment such that the elements tend to be and remain bonded during normal use conditions of the absorbent garment.

“Polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries.

“Refastenable” refers to the property of two elements being capable of releasable attachment, separation, and subsequent releasable reattachment without substantial permanent deformation or rupture.

“Releasably attached,” “releasably engaged,” and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment. It should be noted that a releasably attached or releasably engaged seam is a refastenable seam that does not include a bonded seam that must be torn, cut, or otherwise disrupted.

“Spunbonded fiber” refers to small diameter fibers that are formed by extruding molten thermoplastic material as filaments from a plurality of fine capillaries of a spinnerette having a circular or other configuration, with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein in its entirety by reference. Spunbond fibers are quenched and generally not tacky when they are deposited onto a collecting surface. Spunbond fibers are generally continuous and often have average deniers larger than about 0.3, more particularly, between about 0.6 and 10.

“Stretchable” means that a material can be stretched, without breaking, to at least 150% of its initial (unstretched) length in at least one direction, suitably to at least 200% of its initial length, desirably to at least 250% of its initial length.

“Superabsorbent” or “superabsorbent material” refers to a water-swellable, water-insoluble organic or inorganic material capable, under the most favorable conditions, of absorbing at least about 15 times its weight and, more desirably, at least about 30 times its weight in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds such as cross-linked polymers.

“Surface” includes any layer, film, woven, nonwoven, laminate, composite, or the like, whether pervious or impervious to air, gas, and/or liquids.

“Thermoplastic” describes a material that softens when exposed to heat and that substantially returns to a nonsoftened condition when cooled to room temperature.

These terms can be defined with additional language in the remaining portions of the specification.

### DETAILED DESCRIPTION

The present disclosure is directed to an absorbent article including a closed cell foam matrix for delivering oxygen. Desirably, the absorbent article includes a liquid permeable inner surface for facing the wearer, an outer surface for facing away from the wearer, an absorbent assembly disposed therebetween, and a closed-cell foam matrix material applied to a portion of the absorbent assembly. The term absorbent article generally refers to articles that can be placed against or in proximity to the body of the wearer to absorb and/or retain various liquid wastes discharged from the body. The absorbent article can be disposable, which refers to articles that are intended to be discarded after a limited period of use instead of being laundered or otherwise restored for reuse. It is understood that the concepts described herein are suitable for use with various other pants-type articles such as adult incontinence articles, as well as other articles intended for personal wear such as clothing, diapers, feminine hygiene products such as liners and pads, medical garments, surgical pads and bandages, other personal care or health care garments, and the like without departing from the scope of the present disclosure.

The closed cell foam matrix contains a superabsorbent material and oxygen entrapped within the superabsorbent material. To produce the closed cell foam matrix for delivering oxygen, an alkali hydroxide catalyst and oxygen precursor are both added to an aqueous superabsorbent material. The aqueous solution is applied to a nonwoven material. The nonwoven material is then heated to produce oxygen by reacting the alkali hydroxide catalyst and the oxygen precursor thereby entrapping the oxygen in the formed closed cell foam matrix.

The closed cell foam could be formed in a variety of shapes and forms; such as, in a sheet or layer; coating infused on to a nonwoven matrix; extruded fibers; coating on fibers, powder. All of these forms would be capable of releasing oxygen.

The superabsorbent material has at least 15 percent by mass monoethylenically unsaturated carboxylic, sulphonic or phosphoric acid or salts thereof, an acrylate or methacrylate ester that contains an alkoxysilane functionality, and a copolymerizable hydrophilic glycol containing ester monomer.

The substrate including the oxygen releasing closed cell foam matrix may be incorporated into an absorbent article. Various methods for constructing an absorbent article are described in U.S. patent application Ser. No. 14/062,278 filed Oct. 24, 2013 by Ruman et al.; U.S. patent application Ser. No. 14/068,918 filed Oct. 31, 2013 by Sina et al.; U.S. patent application Ser. No. 14/068,913 filed Oct. 31, 2013 by Bennett et al.; PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al., which are incorporated herein by reference.

Absorbent articles that release oxygen may a myriad of benefits. By releasing oxygen, the product feels more breathable to the user, and imparts a more fresh feeling. Additionally, the articles and compositions using the oxygen releasing closed cell foam matrix could deliver additional benefits beyond oxygen release in an absorbent article. For example, the oxygen releasing closed cell foam matrix could absorb moisture and humidity making the liner more comfortable during wear. The possibility of higher oxygen atmosphere could cause malodor producing bacteria to switch over to produce carbon dioxide, thus reduce odor. A significant advantage of this oxygen releasing closed cell foam matrix is its process-friendliness. The oxygen releasing closed cell foam matrix could be easily coated onto any conventional nonwovens material for application into the product.

Restoring oxygen microclimate balance or possibly slightly in excess would potentially reduce skin irritation, reduce pathogenic bacteria and yeasts, speed up the recovery of damaged skin (e.g. irritation or dermatitis).

As discussed above, the closed cell foam is produced with a superabsorbent polymer material. A superabsorbent polymer material suitable for use herein is described as a superabsorbent binder polymer solution in U.S. Pat. No. 6,849,685 to Soerens et al., U.S. Pat. No. 7,312,286 to Lang et al., and U.S. Pat. No. 7,335,713 to Lang et al., the entirety of each of these references is herein incorporated by reference. The superabsorbent binder polymer solution described therein is capable of post-application, moisture-induced crosslinking. Whereas most superabsorbent polymers require the addition of an internal crosslinker to reinforce the polymer, the superabsorbent polymer material used herein does not require the addition of a crosslinking agent because the organic monomers act as an internal crosslinker. The internal crosslinker allows the superabsorbent polymer material to be formed by coating the water-soluble precursor polymer onto the substrate and then removing the water to activate the latent crosslinker.

Soerens et al., in U.S. Pat. No. 6,737,491, describes an absorbent binder composition that may be used as a superabsorbent polymer material described herein. The absorbent binder composition disclosed in Soerens et al. is a monoethylenically unsaturated polymer and an acrylate or methacrylate ester that contains an alkoxysilane functionality that is particularly suitable for use in manufacturing absorbent articles. Also described in Soerens et al. is a method of making the absorbent binder composition that includes the steps of preparing a monomer solution, adding the monomer solution to an initiator system, and activating a polymerization initiator within the initiator system reported an alcohol-based, water-soluble binder composition. “Monomer(s)” as used herein includes monomers, oligomers, polymers, mixtures of monomers, oligomers and/or polymers, and any other reactive chemical species which are capable of co-polymerization with monoethylenically unsaturated carboxylic, sulphonic or phosphoric acid or salts thereof. Ethylenically unsaturated monomers containing a trialkoxysilane functional group are appropriate for this invention and are desired. Desired ethylenically unsaturated monomers include acrylates and methacrylates, such as acrylate or methacrylate esters that contain an alkoxysilane functionality.

The superabsorbent binder polymer composition disclosed in the references noted above is the reaction product of at least 15 percent by mass monoethylenically unsaturated carboxylic, sulphonic or phosphoric acid or salts thereof, an acrylate or methacrylate ester that contains an alkoxysilane functionality which, upon exposure to water, forms a silanol functional group which condenses to form a crosslinked polymer, a copolymerizable hydrophilic glycol containing ester monomer; and/or, a plasticizer.

The monoethylenically unsaturated monomer is desirably acrylic acid. Other suitable monomers include carboxyl group-containing monomers: for example monoethylenically unsaturated mono or poly-carboxylic acids, such as (meth)acrylic acid (meaning acrylic acid or methacrylic acid; similar notations are used hereinafter), maleic acid, fumaric acid, crotonic acid, sorbic acid, itaconic acid, and cinnamic acid; carboxylic acid anhydride group-containing monomers: for example monoethylenically unsaturated polycarboxylic acid anhydrides (such as maleic anhydride); carboxylic acid salt-containing monomers: for example water-soluble salts (alkali metal salts, ammonium salts, amine salts, and the like) of monoethylenically unsaturated mono- or poly-carboxylic acids (such as sodium (meth)acrylate, trimethylamine (meth)acrylate, triethanolamine (meth)acrylate), sodium maleate, methylamine maleate; sulfonic acid group-containing monomers: for example aliphatic or aromatic vinyl sulfonic acids (such as vinylsulfonic acid, allyl sulfonic acid, vinyltoluenesulfonic acid, styrene sulfonic acid), (meth)acrylic sulfonic acids [such as sulfopropyl (meth)acrylate, 2-hydroxy-3-(meth)acryloxy propyl sulfonic acid]; sulfonic acid salt group-containing monomers: for example alkali metal salts, ammonium salts, amine salts of sulfonic acid group containing monomers as mentioned above; and/or amide group-containing monomers: vinylformamide, (meth)acrylamide, N-alkyl (meth)acrylamides (such as N-methylacrylamide, N-hexylacrylamide), N,N-dialkyl (meth)acryl amides (such as N,N-dimethylacrylamide, N,N-di-n-propylacrylamide), N-hydroxyalkyl (meth)acrylamides [such as N-methylol (meth)acrylamide, N-hydroxyethyl (meth)acrylamide], N,N-dihydroxyalkyl (meth)acrylamides [such as N,N-dihydroxyethyl (meth)acrylamide], vinyl lactams (such as N-vinylpyrrolidone).

Suitably, the amount of monoethylenically unsaturated carboxylic, sulphonic or phosphoric acid or salts thereof relative to the weight of the superabsorbent binder polymer composition may range from about 15 percent to about 99.9 percent by weight. The acid groups are desirably neutralized to the extent of at least about 25 mol percent, that is, the acid groups are preferably present as sodium, potassium or ammonium salts. The degree of neutralization is preferably at least about 50 mol percent.

One of the issues in preparing water-soluble polymers is the amount of the residual monoethylenically unsaturated monomer content remaining in the polymer. For applications in personal hygiene it is required the amount of residual monoethylenically unsaturated monomer content of the superabsorbent polymer composition be less than about 1000 ppm, and more preferably less than 500 ppm, and even more preferably less than 100 ppm. U.S. Pat. No. 7,312,286 discloses at least one method by which an absorbent binder composition may be manufactured so that the residual monoethylenically unsaturated monomer content is at least less than 1000 parts per million. The analysis of residual monoethylenically unsaturated monomer is determined according to the Residual Monoethylenically Unsaturated Monomer Test which is disclosed in U.S. Pat. No. 7,312,286. More specifically, the residual monoethylenically unsaturated monomer analysis is carried out using solid film obtained from the polymer solution or superabsorbent composition. By way of example for this test description, the monoethylenically unsaturated monomer is acrylic acid. High performance liquid chromatography (HPLC) with a SPD-IOAvp Shimadzu UV detector (available from Shimadzu Scientific Instruments, having a place of business in Columbia, Md., U.S.A.) is used to determine the residual acrylic acid monomer content. To determine the residual acrylic acid monomer, about 0. 5 grams of cured film is stirred in 100 ml of a 0.9% NaCI-solution for 16 h using a 3.5 cm L×0.5 cm W magnetic stirrer bar at 500 rpm speed. The mixture is filtered and the filtrate is then passed through a Nucleosil C8 100A reverse phase column (available from Column Engineering Incorporated, a business having offices located in Ontario, Calif., U.S.A.) to separate the acrylic acid monomer. The acrylic acid monomer elutes at a certain time with detection limit at about 10 ppm. The peak area of resulting elutes calculated from the chromatogram is then used to calculate the amount of residual acrylic acid monomer in the film. Initially, a calibration curve was generated by plotting the response area of pure acrylic acid elutes against its known amount (ppm). A linear curve with a correlation coefficient of greater than 0.996 was obtained.

Desirably, an aqueous solution of an oligomeric polyacrylic acid having a silanol cross-linker covalently bonded to the backbone chain of a polyacrylic acid is used for the superabsorbent material described herein.

To produce the closed cell foam matrix for delivering oxygen, an alkali hydroxide catalyst and an oxygen precursor is added to an aqueous solution containing the superabsorbent material. Examples of alkali hydroxide catalyst that can be used include, but are not limited to, sodium hydroxide, lithium hydroxide, potassium hydroxide, magnesium hydroxide, calcium hydroxide, and combinations thereof. In desirable embodiments, the alkali hydroxide catalyst comprises sodium hydroxide. Suitably, the amount of the alkali hydroxide catalyst that is added may be between about 0.5 percent to about 3 percent by weight relative to the weight of the liquid superabsorbent polymer composition.

Examples of the oxygen presursor that can be used include, but are not limited to, hydrogen peroxide, ammonium peroxide, sodium peroxide, urea-peroxide, potassium percarbonate, and combinations thereof. In desirable embodiments, the oxygen precursor comprises hydrogen peroxide. Suitably, the amount of the oxygen precursor that is added may be between about 15 percent to about 25 percent by weight relative to the weight of the liquid superabsorbent polymer composition.

In another embodiment, a molar ratio of the alkali hydroxide catalyst to the oxygen precursor is in the range of 1.0:0.9 to 0.9:1.0 with the alkalki hydroxide catalyst having an additional amount to neutralize the acid component superabsorbent material.

Any suitable dispensing means, such as a spray nozzle, doctor blade, roller applicator, dip & squeeze or the like, may be used to apply an aqueous solution containing the superabsorbent, oxygen precursor and alkali hydrogen catalyst to the surface of a substrate. A vacuum applied by suction box positioned beneath the dispensing means helps to draw the absorbent composite stabilizer into the substrate. The dispensing means or applicator is essentially coextensive with the width of the substrate, and preferably a substantially uniform coating of the aqueous solution is applied to the substrate. However, the aqueous solution may be applied as a non-uniform, random or pattern coating, and because the aqueous solution is water-based, it will diffuse throughout the substrate. The extent or degree of penetration of the aqueous solution into the substrate may be controlled by controlling the amount of aqueous solution applied and by controlling the vacuum applied to the substrate. Other configurations for applying the aqueous solution to the substrate can be used by a person skilled in the art.

The substrate and applied aqueous solution containing the superabsorbent, oxygen precursor and alkali hydrogen catalyst is then heated to produce oxygen by reacting the alkali hydroxide catalyst and the oxygen precursor thereby entrapping the oxygen in a formed closed cell foam matrix. In preferred embodiments, the substrate is heated at a temperature of at least 50 degrees Celsius.

As described above, the substrate including the formed closed cell foam matrix may be applied as a portion of an absorbent article. For example, the formed closed cell foam matrix may be applied to an absorbent body, an outer cover layer, an acquisition layer, a transfer layer, a core wrap, waistband or any other layer on an absorbent article. The foamed close cell foam matrix could also be used as a fiber or a particulate powder or a fiber coating. Various methods for constructing an absorbent article are described in U.S. patent application Ser. No. 14/062,278 filed Oct. 24, 2013 by Ruman et al.; U.S. patent application Ser. No. 14/068,918 filed Oct. 31, 2013 by Sina et al.; U.S. patent application Ser. No. 14/068,913 filed Oct. 31, 2013 by Bennett et al.; PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al., which are incorporated herein by reference.

Referring to FIG. 1, a disposable absorbent article 110 of the present disclosure is exemplified in the form of a diaper. It is to be understood that the present disclosure is suitable for use with various other personal care absorbent articles, such as, for example, feminine hygiene products, adult incontinence products and pads, without departing from the scope of the present disclosure. While the embodiments and illustrations described herein may generally apply to absorbent articles manufactured in the product longitudinal direction, which is hereinafter called the machine direction manufacturing of a product, it should be noted that one of ordinary skill could apply the information herein to absorbent articles manufactured in the latitudinal direction of the product which hereinafter is called the cross direction manufacturing of a product without departing from the spirit and scope of the disclosure. The absorbent article 110 illustrated in FIG. 1 includes a front waist region 112, a back waist region 114, and a crotch region 116 interconnecting the front and back waist regions, 112 and 114, respectively. The absorbent article 110 has a pair of longitudinal side edges, 112 and 114 (shown in FIG. 2), and a pair of opposite waist edges, respectively designated front waist edge 122 and back waist edge 124. The front waist region 112 can be contiguous with the front waist edge 122 and the back waist region 114 can be contiguous with the back waist edge 124.

Referring to FIG. 2, a non-limiting illustration of an absorbent article 110, such as, for example, a diaper, is illustrated in a top down view with portions cut away for clarity of illustration. The absorbent article 110 can include an outer cover 126 and a body facing material 128. In an embodiment, the body facing material 128 can be bonded to the outer cover 126 in a superposed relation by any suitable means such as, but not limited to, adhesives, ultrasonic bonds, thermal bonds, pressure bonds, or other conventional techniques. The outer cover 126 can define a length, or longitudinal direction 130, and a width, or lateral direction 132, which, in the illustrated embodiment, can coincide with the length and width of the absorbent article 110. The longitudinal direction 130 and the lateral direction 132 of the absorbent article 110, and of the materials which form the absorbent article 110, can provide the X-Y planes, respectively, of the absorbent article 110 and of the materials which form the absorbent article 110. The absorbent article 110, and the materials which form the absorbent article 110, can also have a Z-direction. A measurement, taken under pressure, in the Z-direction of a material which forms the absorbent article 110 can provide a measurement of the thickness of the material. A measurement, taken under pressure, in the Z-direction of the absorbent article 110 can provide a measurement of the bulk of the absorbent article 110.

Referring to FIGS. 1-6, an absorbent body 140 can be disposed between the outer cover 126 and the body facing material 128. The absorbent body 140 can have longitudinal edges, 142 and 144, which, in an embodiment, can form portions of the longitudinal side edges, 118 and 120, respectively, of the absorbent article 110 and can have opposite end edges, 146 and 148, which, in an embodiment, can form portions of the waist edges, 122 and 124, respectively, of the absorbent article 110. In an embodiment, the absorbent body 140 can have a length and width that are the same as or less than the length and width of the absorbent article 110. In an embodiment, a pair of containment flaps, 150 and 152, can be present and can inhibit the lateral flow of body exudates.

The front waist region 112 can include the portion of the absorbent article 110 that, when worn, is positioned at least in part on the front of the wearer while the back waist region 114 can include the portion of the absorbent article 110 that, when worn, is positioned at least in part on the back of the wearer. The crotch region 116 of the absorbent article 110 can include the portion of the absorbent article 110, that, when worn, is positioned between the legs of the wearer and can partially cover the lower torso of the wearer. The waist edges, 122 and 124, of the absorbent article 110 are configured to encircle the waist of the wearer and together define the central waist opening 154 (such as shown in FIG. 1). Portions of the longitudinal side edges, 118 and 120, in the crotch region 116 can generally define leg openings 156 (such as shown in FIG. 1) when the absorbent article 110 is worn.

The absorbent article 110 can be configured to contain and/or absorb liquid, solid, and semi-solid body exudates discharged from the wearer. For example, containment flaps, 150 and 152, can be configured to provide a barrier to the lateral flow of body exudates. A flap elastic member, 158 and 160, can be operatively joined to each containment flap, 150 and 152, in any suitable manner known in the art. The elasticized containment flaps, 150 and 152, can define a partially unattached edge that can assume an upright configuration in at least the crotch region 116 of the absorbent article 110 to form a seal against the wearer's body. The containment flaps, 150 and 152, can be located along the absorbent article 110 longitudinal side edges, 118 and 120, and can extend longitudinally along the entire length of absorbent article 110 or can extend partially along the length of the absorbent article 110. Suitable construction and arrangements for containment flaps, 150 and 152, are generally well known to those skilled in the art and are described in U.S. Pat. No. 4,704,116 issued Nov. 3, 1987, to Enloe and U.S. Pat. No. 5,562,650 issued Oct. 8, 1996 to Everett et al., which are incorporated herein by reference.

To further enhance containment and/or absorption of body exudates, the absorbent article 110 can suitably include a front waist elastic member 162, a rear waist elastic member 164, and leg elastic members, 166 and 168, as are known to those skilled in the art. The waist elastic members, 162 and 164, can be attached to the outer cover 126, the body facing material 128 along the opposite waist edges, 122 and 124, and can extend over part or all of the waist edges, 122 and 124. The leg elastic members, 166 and 168, can be attached to the outer cover 126, the body facing material 128 along the opposite longitudinal side edges, 118 and 120, and positioned in the crotch region 116 of the absorbent article 110.

Additional details regarding each of these elements of the absorbent article 110 described herein can be found below and with reference to the Figures.

Outer Cover:

The outer cover 126 can be breathable and/or liquid impermeable. The outer cover 126 can be elastic, stretchable or non-stretchable. The outer cover 126 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer cover 126 can be constructed of a microporous polymeric film, such as polyethylene or polypropylene.

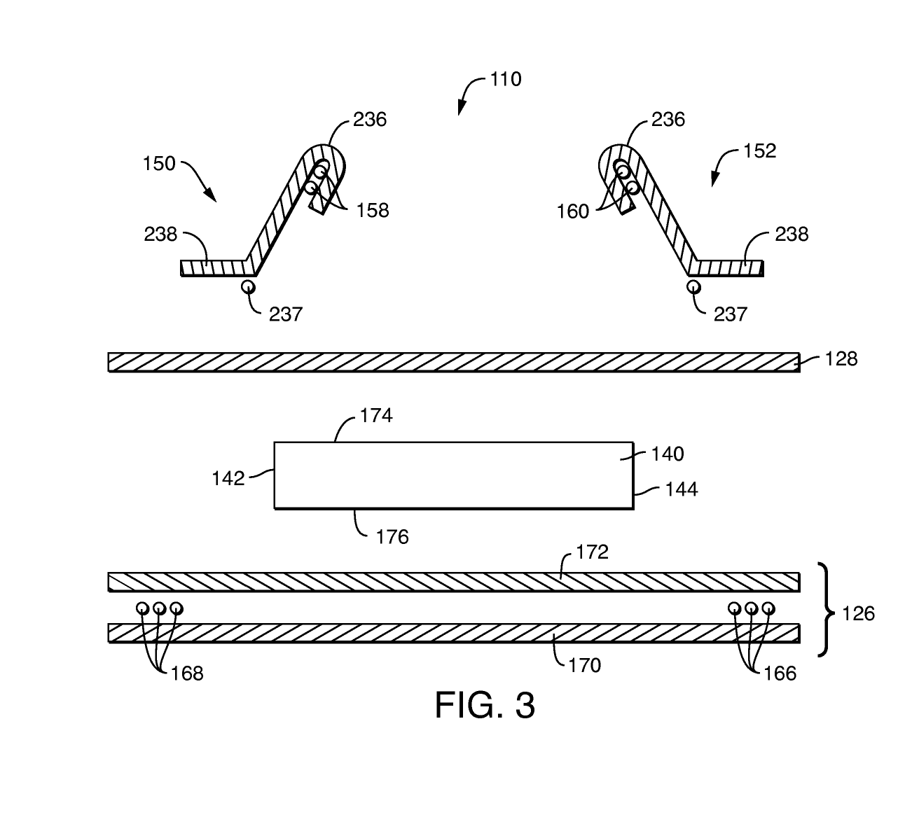

In an embodiment, the outer cover 126 can be a single layer of a liquid impermeable material. In an embodiment, the outer cover 126 can be suitably stretchable, and more suitably elastic, in at least the lateral or circumferential direction 132 of the absorbent article 110. In an embodiment, the outer cover 126 can be stretchable, and more suitably elastic, in both the lateral 132 and the longitudinal 130 directions. In an embodiment, the outer cover 126 can be a multi-layered laminate in which at least one of the layers is liquid impermeable. In an embodiment such as illustrated in FIGS. 3-6, the outer cover 126 may be a two layer construction, including an outer layer 170 material and an inner layer 172 material which can be bonded together such as by a laminate adhesive. Suitable laminate adhesives can be applied continuously or intermittently as beads, a spray, parallel swirls, or the like. Suitable adhesives can be obtained from Bostik Findlay Adhesives, Inc. of Wauwatosa, Wis., U.S.A. It is to be understood that the inner layer 172 can be bonded to the outer layer 170 utilizing ultrasonic bonds, thermal bonds, pressure bonds, or the like.

The outer layer 170 of the outer cover 126 can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer 170 of an outer cover 126 can be a 20 gsm spunbond polypropylene non-woven web.

The liquid impermeable inner layer 172 of the outer cover 126 (or the liquid impermeable outer cover 126 where the outer cover 126 is of a single-layer construction) can be either vapor permeable (i.e., “breathable”) or vapor impermeable. The liquid impermeable inner layer 172 (or the liquid impermeable outer cover 126 where the outer cover 126 is of a single-layer construction) may be manufactured from a thin plastic film, although other liquid impermeable materials may also be used. The liquid impermeable inner layer 172 (or the liquid impermeable outer cover 126 where the outer cover 126 is of a single-layer construction) can inhibit liquid body exudates from leaking out of the absorbent article 110 and wetting articles, such as bed sheets and clothing, as well as the wearer and caregiver. An example of a material for a liquid impermeable inner layer 172 (or the liquid impermeable outer cover 126 where the outer cover 126 is of a single-layer construction) can be a printed 19 gsm Berry Plastics XP-8695H film or equivalent commercially available from Berry Plastics Corporation, Evansville, Ind., U.S.A.

Where the outer cover 126 is of a single layer construction, it can be embossed and/or matte finished to provide a more cloth-like texture or appearance. The outer cover 126 can permit vapors to escape from the absorbent article 110 while preventing liquids from passing through. A suitable liquid impermeable, vapor permeable material can be composed of a microporous polymer film or a non-woven material which has been coated or otherwise treated to impart a desired level of liquid impermeability.

As described above, the formed closed cell foam matrix may be applied to the outercover 126.

Absorbent Body:

The absorbent body 140 can be superposed over the inner layer 172 of the outer cover 126, extending laterally between the leg elastic members, 166 and 168, and can be bonded to the inner layer 172 of the outer cover 126, such as by being bonded thereto with adhesive. However, it is to be understood that the absorbent body 140 may be in contact with, and not bonded with, the outer cover 126 and remain within the scope of this disclosure. In an embodiment, the outer cover 126 can be composed of a single layer and the absorbent body 140 can be in contact with the single layer of the outer cover 126. In an embodiment, a layer, such as but not limited to, a core wrap layer 178, can be positioned between the absorbent body 140 and the outer cover 126. As described above, the formed closed cell foam matrix may be applied to the absorbent body 140.

Core Wrap Layer:

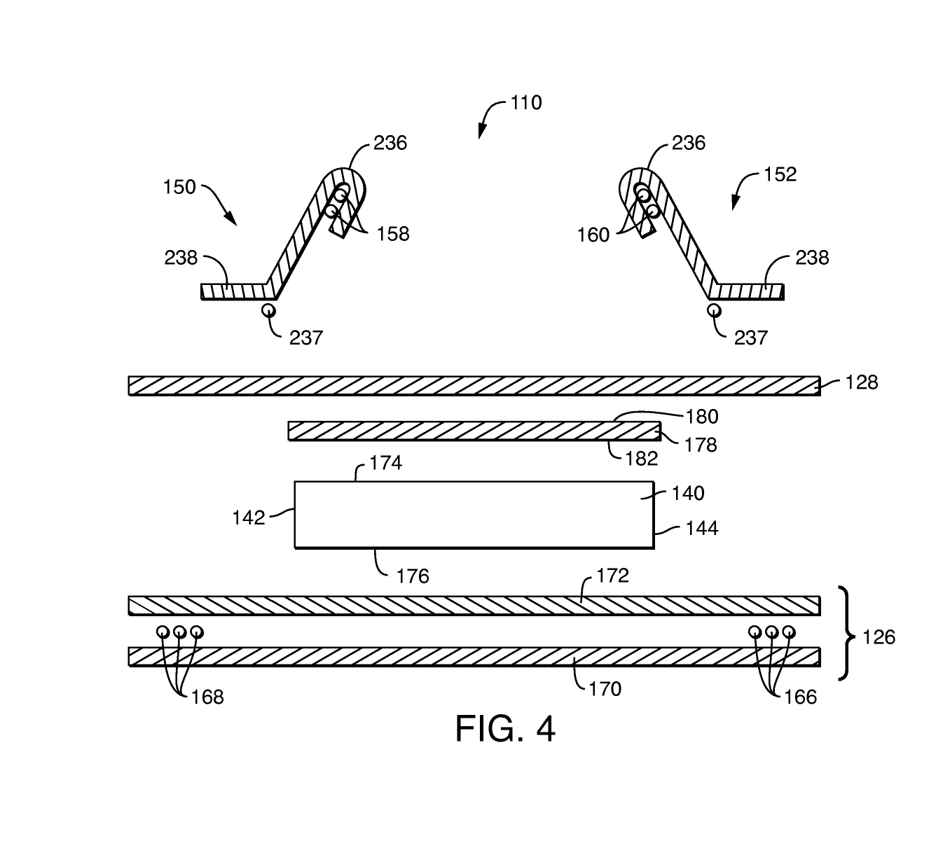

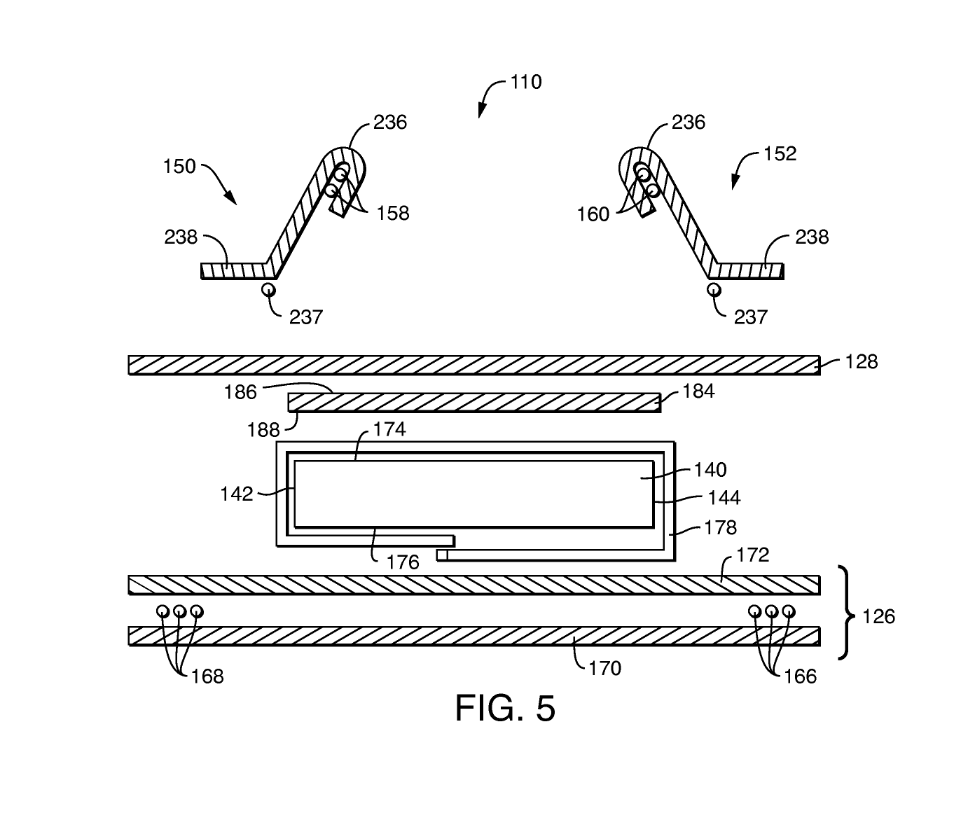

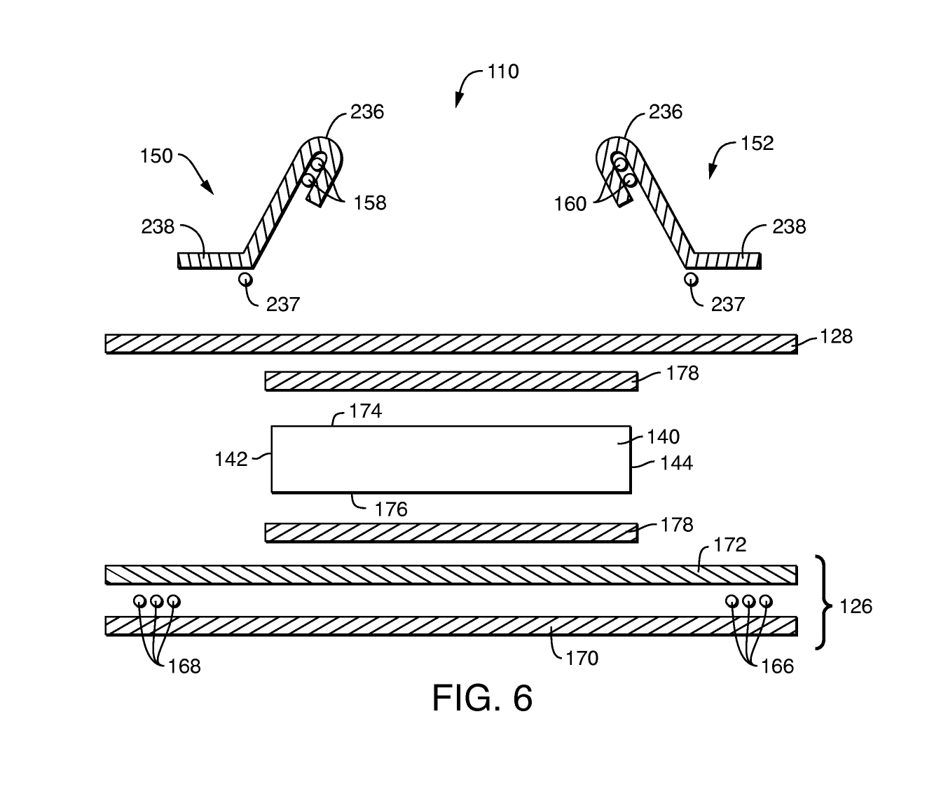

In various embodiments, such as illustrated in the non-limiting example of FIG. 3, an absorbent article 110 can be constructed without a core wrap layer 178. In various embodiments, such as illustrated in the non-limiting examples of FIGS. 4-6, the absorbent article 110 can have a core wrap layer 178. The core wrap layer 178 can have a wearer facing surface 174 and a garment facing surface 176. In an embodiment, the core wrap layer 178 can be in contact with the absorbent body 140. In an embodiment, the core wrap layer 178 can be bonded to the absorbent body 140. Bonding of the core wrap layer 178 to the absorbent body 140 can occur via any means known to one of ordinary skill, such as, but not limited to, adhesives. In an embodiment, such as illustrated in the non-limiting example of FIG. 4, a core wrap layer 178 can be positioned between the body facing material 128 and the absorbent core 140. In an embodiment, such as illustrated in the non-limiting example of FIG. 5, a core wrap layer 178 can completely encompass the absorbent body 140 and can be sealed to itself. In such an embodiment, the core wrap layer 178 may be folded over on itself and then sealed using, for example, heat, adhesive and/or pressure. In an embodiment, such as, for example, in the non-limiting illustration of FIG. 6, a core wrap layer 178 may be composed of separate sheets of material which can be utilized to partially or fully encompass the absorbent body 140 and which can be sealed together using a sealing means such as an ultrasonic bonder or other thermochemical bonding means or the use of an adhesive. In another embodiment, there is no core wrap layer at all. In other embodiments, the core wrap layer can be on only one of the wearer facing surface 174 and a garment facing surface 176. Due the nature of the absorbent structure defined herein, the core wrap may not be included on the wearer facing surface due to the increased integrity of absorbent structure resulting in less gel on skin issues. Due the nature of the absorbent structure defined herein, the core wrap may not be included on the garment facing surface, due to the absorbent structure swelling and removing soft edges.

In an embodiment, the core wrap layer 178 can be in contact with and/or bonded with the wearer facing surface 174 of the absorbent body 140. In an embodiment, the core wrap layer 178 can be in contact with and/or bonded with the wearer facing surface 174 and at least one of the edges, 142, 144, 146 and/or 148, of the absorbent body 140. In an embodiment, the core wrap layer 178 can be in contact with and/or bonded with the wearer facing surface 174, at least one of the edges, 142, 144, 146 and/or 148, and the garment facing surface 176 of the absorbent body 140. In an embodiment, the absorbent body 140 may be partially or completely encompassed by a core wrap layer 178.

The core wrap layer 178 can be pliable, less hydrophilic than the absorbent body 140, and sufficiently porous to thereby permit liquid body exudates to penetrate through the core wrap layer 178 to reach the absorbent body 140. In an embodiment, the core wrap layer 178 can have sufficient structural integrity to withstand wetting thereof and of the absorbent composite. In an embodiment, the core wrap layer 178 can be constructed from a single layer of material or it may be a laminate constructed from two or more layers of material.

In an embodiment, the core wrap layer 178 can include, but is not limited to, natural and synthetic fibers such as, but not limited to, polyester, polypropylene, acetate, nylon, polymeric materials, cellulosic materials such as wood pulp, cotton, rayon, viscose, LYOCELL® such as from Lenzing Company of Austria, or mixtures of these or other cellulosic fibers, and combinations thereof. Natural fibers can include, but are not limited to, wool, cotton, flax, hemp, and wood pulp.

In various embodiments, the core wrap layer selected from metlblown-spunbond-meltblown fabric, spunbond fabric, meltblown fabric, coform fabric, carded web, bonded-carded web, bicomponent spunbond fabric, spunlace, tissue, and combinations thereof.

In various embodiments, the core wrap layer 178 can include cellulosic material. In various embodiments, the core wrap layer 178 can be creped wadding or a high-strength tissue. In various embodiments, the core wrap layer 178 can include polymeric material. In an embodiment, a core wrap layer 178 can include a spunbond material. In an embodiment, a core wrap layer 178 can include a meltblown material. In an embodiment, the core wrap layer 178 can be a laminate of a meltblown nonwoven material having fine fibers laminated to at least one spunbond nonwoven material layer having coarse fibers. In such an embodiment, the core wrap layer 178 can be a spunbond-meltblown (“SM”) material. In an embodiment, the core wrap layer 178 can be a spunbond-meltblown-spunbond (“SMS”) material. A non-limiting example of such a core wrap layer 178 can be a 10 gsm spunbond-meltblown-spunbond material. In various embodiments, the core wrap layer 178 can be composed of at least one material which has been hydraulically entangled into a nonwoven substrate. In various embodiments, the core wrap layer 178 can be composed of at least two materials which have been hydraulically entangled into a nonwoven substrate. In various embodiments, the core wrap layer 178 can have at least three materials which have been hydraulically entangled into a nonwoven substrate. A non-limiting example of a core wrap layer 178 can be a 33 gsm hydraulically entangled substrate. In such an example, the core wrap layer 178 can be a 33 gsm hydraulically entangled substrate composed of a 12 gsm spunbond material, a 10 gsm wood pulp material having a length from about 0.6 cm to about 5.5 cm, and an 11 gsm polyester staple fiber material. To manufacture the core wrap layer 178 just described, the 12 gsm spunbond material can provide a base layer while the 10 gsm wood pulp material and the 11 gsm polyester staple fiber material can be homogeneously mixed together and deposited onto the spunbond material and then hydraulically entangled with the spunbond material.

In various embodiments, a wet strength agent can be included in the core wrap layer 178. A non-limiting example of a wet strength agent can be Kymene 6500 (557LK) or equivalent available from Ashland Inc. of Ashland, Ky., U.S.A. In various embodiments, a surfactant can be included in the core wrap layer 178. In various embodiments, the core wrap layer 178 can be hydrophilic. In various embodiments, the core wrap layer 178 can be hydrophobic and can be treated in any manner known in the art to be made hydrophilic.

In an embodiment, the core wrap layer 178 can be in contact with and/or bonded with an absorbent composite which is made at least partially of particulate material such as superabsorbent material. In an embodiment in which the core wrap layer 178 at least partially or completely encompasses the absorbent body 140.

In an embodiment, the core wrap layer 178 may have a longitudinal length the same as, greater than, or less than the longitudinal length of the absorbent composite 140. The core wrap layer 178 can have a longitudinal length ranging from about 150 to about 520 mm.

As described above, the formed closed cell foam matrix may be applied to the core wrap layer 178.

Acquisition Layer:

In various embodiments, such as illustrated, for example, in FIG. 5, the absorbent article 10 can have an acquisition layer 184. The acquisition layer 184 can help decelerate and diffuse surges or gushes of liquid body exudates penetrating the body facing material 128. In an embodiment, the acquisition layer 184 can be positioned between the body facing material 128 and the absorbent body 140 to take in and distribute body exudates for absorption by the absorbent body 140. In an embodiment, the acquisition layer 184 can be positioned between the body facing material 128 and a core wrap layer 178 if a core wrap layer 178 is present.

The acquisition layer 184 can have a wearer facing surface 186 and a garment facing surface 188. In an embodiment, the acquisition layer 184 can be in contact with and/or bonded with the body facing material 128. In an embodiment in which the acquisition layer 184 is bonded with the body facing material 128, bonding of the acquisition layer 184 to the body facing material 128 can occur through the use of an adhesive and/or point fusion bonding. The point fusion bonding can be selected from, but is not limited to, ultrasonic bonding, pressure bonding, thermal bonding, and combinations thereof. In an embodiment, the point fusion bonding can be provided in any pattern as deemed suitable.

The acquisition layer 184 may have any longitudinal length dimension as deemed suitable. In an embodiment, the acquisition layer 184 can have any length such that the acquisition layer 184 can be coterminous with the waist edges, 122 and 124, of the absorbent article 110.

In an embodiment, the longitudinal length of the acquisition layer 184 can be the same as the longitudinal length of the absorbent body 140. In such an embodiment the midpoint of the longitudinal length of the acquisition layer 184 can substantially align with the midpoint of the longitudinal length of the absorbent body 140.

In an embodiment, the longitudinal length of the acquisition layer 184 can be shorter than the longitudinal length of the absorbent body 140. In such an embodiment, the acquisition layer 184 may be positioned at any desired location along the longitudinal length of the absorbent body 140. As an example of such an embodiment, the absorbent article 110 may contain a target area where repeated liquid surges typically occur in the absorbent article 110. The particular location of a target area can vary depending on the age and gender of the wearer of the absorbent article 110 and design of the absorbent article 110. For example, males tend to urinate further toward the front region of the absorbent article 110 and the target area may be phased forward within the absorbent article 110. For example, the target area for a male wearer may be positioned about 70 mm forward of the longitudinal midpoint of the absorbent composite. The female target area can be located closer to the center of the crotch region 116 of the absorbent article 110. For example, the target area for a female wearer may be positioned about 26 mm forward of the longitudinal midpoint of the absorbent body 140. As a result, the relative longitudinal placement of the acquisition layer 184 within the absorbent article 110 can be selected to best correspond with the target area of either or both categories of wearers.

In an embodiment, the acquisition layer 184 can have a size dimension that is the same size dimension as the target area of the absorbent article 110 or a size dimension greater than the size dimension of the target area of the absorbent article 110. In an embodiment, the acquisition layer 184 can be in contact with and/or bonded with the body facing material 128 at least partially in the target area of the absorbent article 110.

In various embodiments, the acquisition layer 184 can have a longitudinal length shorter than, the same as or longer than the longitudinal length of the absorbent body 140. In an embodiment in which the absorbent article 110 is a diaper, the acquisition layer 184 may have a longitudinal length from about 120 to about 320 mm. In such an embodiment, the acquisition layer 184 may be shorter in longitudinal length than the longitudinal length of the absorbent body 140 and may be phased from the front end edge 146 of the absorbent body 140 a distance of from about 15 mm to about 40 mm. In an embodiment in which the absorbent article 110 may be a training pant or youth pant, the acquisition layer 184 may have a longitudinal length from about 120 to about 520 mm. In such an embodiment, the acquisition layer 184 may have a longitudinal length shorter than the longitudinal length of the absorbent body 140 and may be phased a distance of from about 25 mm to about 85 mm from the front end edge 146 of the absorbent composite 140. In an embodiment in which the absorbent article 110 is an adult incontinence garment, the acquisition layer 184 may have a longitudinal length from about 200 mm to about 450 mm. In such an embodiment, the acquisition layer 184 may have a longitudinal length shorter than the longitudinal length of the absorbent body 140 and the acquisition layer 184 may be phased a distance of from about 20 mm to about 75 mm from the front end edge 146 of the absorbent body 140.

The acquisition layer 184 may have any width as desired. The acquisition layer 184 may have a width dimension from about 15 mm to about 180 mm. The width of the acquisition layer 184 may vary dependent upon the size and shape of the absorbent article 110 within which the acquisition layer 184 will be placed. The acquisition layer 184 can have a width smaller than, the same as, or larger than the width of the absorbent body 140. Within the crotch region 116 of the absorbent article 110, the acquisition layer 184 can have a width smaller than, the same as, or larger than the width of the absorbent body 140.

In an embodiment, the acquisition layer 184 can include natural fibers, synthetic fibers, superabsorbent material, woven material, nonwoven material, wet-laid fibrous webs, a substantially unbounded airlaid fibrous web, an operatively bonded, stabilized-airlaid fibrous web, or the like, as well as combinations thereof. In an embodiment, the acquisition layer 184 can be formed from a material that is substantially hydrophobic, such as a nonwoven web composed of polypropylene, polyethylene, polyester, and the like, and combinations thereof.

In various embodiments, the acquisition layer 184 can have fibers which can have a denier of greater than about 5. In various embodiments, the acquisition layer 184 can have fibers which can have a denier of less than about 5. In various embodiments, the fluid acquisition layer selected from meltblown-spunbond-meltblown fabric, spunbond fabric, meltblown fabric, coform fabric, carded web, bonded-carded web, bicomponent spunbond fabric, spunlace, tissue, and combinations thereof

In an embodiment, the acquisition layer 184 can be a through-air bonded-carded web such as a 50 gsm through-air bonded-carded web composite having a homogenous blend of about 50% sheath/core bicomponent polyethylene/polypropylene fibers having a fiber diameter of 3 denier and about 50% sheath/core bicomponent polyethylene/polypropylene fibers having a fiber diameter of 1.5 denier. An example of such a composite is a composite having about 50% ES FiberVisions 3 denier ESC-233 bicomponent fibers and about 50% ES FiberVisions 1.5 denier ESC-215 bicomponent fibers, or equivalent composite, available from ES FiberVisions Corp., Duluth, Ga., U.S.A.

In an embodiment, the acquisition layer 184 can be a through-air bonded-carded web such as a 50 gsm through-air bonded-carded web composite having a homogenous blend of about 50% Rayon fibers having a fiber diameter of 3 denier and about 50% sheath/core bicomponent polyethylene/polypropylene fibers having a fiber diameter of 1.5 denier. An example of such a composite is a composite having about 50% Kelheim 3 denier Rayon Galaxy fibers and about 50% ES FiberVisions 1.5 denier ESC-215 bicomponent fibers, or equivalent composite, available from ES FiberVisions Corp., Duluth, Ga., U.S.A.

In an embodiment, the acquisition layer 184 can be a through-air bonded-carded web such as a 35 gsm through-air bonded-carded web composite having a homogenous mixture of about 35% sheath/core bicomponent polyethylene/polypropylene fibers having a fiber diameter of 6 denier, about 35% sheath/core bicomponent polyethylene/polypropylene fibers having a fiber diameter of 2 denier, and about 30% polyester fibers having a fiber diameter of 6 denier. An example of such a composite is a composite having about 35% Huvis 180-N (PE/PP 6d), about 35% Huvis N-215 (PE/PP 2d), and about 30% Huvis SD-10 PET 6d, or equivalent composite, available from SamBo Company, Ltd, Korea.

In an embodiment, the acquisition layer 184 can be a thermally bonded, stabilized-airlaid fibrous web (e.g. Concert product code DT200.100.D0001) which is available from Glatfelter, a business having offices located in York, Pa., U.S.A.

As described above, the formed closed cell foam matrix may be applied to the acquisition layer 184.

Containment Flaps:

In an embodiment, containment flaps, 150 and 152, can be secured to the body facing material 128 of the absorbent article 110 in a generally parallel, spaced relation with each other laterally inward of the leg openings 156 to provide a barrier against the flow of body exudates to the leg openings 156. In an embodiment, the containment flaps, 150 and 152, can extend longitudinally from the front waist region 112 of the absorbent article 110, through the crotch region 116 to the back waist region 114 of the absorbent article 110. The containment flaps, 150 and 152, can be bonded to the body facing material by a seam of adhesive 237 to define a fixed proximal end 238 of the containment flaps, 150 and 152.

The containment flaps, 150 and 152, can be constructed of a fibrous material which can be similar to the material forming the body facing material 128. Other conventional material, such as polymer films, can also be employed. Each containment flap, 150 and 152, can have a moveable distal end 236 which can include flap elastics, such as flap elastics 158 and 160, respectively. Suitable elastic materials for the flap elastic, 158 and 160, can include sheets, strands or ribbons of natural rubber, synthetic rubber, or thermoplastic elastomeric materials.

The flap elastics, 158 and 160, as illustrated, can have two strands of elastomeric material extending longitudinally along the distal ends 236 of the containment flaps, 150 and 152, in generally parallel, spaced relation with each other. The elastic strands can be within the containment flaps, 150 and 152, while in an elastically contractible condition such that contraction of the strands gathers and shortens the distal ends 236 of the containment flaps, 150 and 152. As a result, the elastic strands can bias the distal ends 236 of each containment flap, 150 and 152, toward a position spaced from the proximal end 238 of the containment flaps, 150 and 152, so that the containment flaps, 150 and 152, can extend away from the body facing material 128 in a generally upright orientation of the containment flaps, 150 and 152, especially in the crotch region 116 of the absorbent article 110, when the absorbent article 110 is fitted on the wearer. The distal end 236 of the containment flaps, 150 and 152, can be connected to the flap elastics, 158 and 160, by partially doubling the containment flap, 150 and 152, material back upon itself by an amount which can be sufficient to enclose the flap elastics, 158 and 160. It is to be understood, however, that the containment flaps, 150 and 152, can have any number of strands of elastomeric material and may also be omitted from the absorbent article 110 without departing from the scope of this disclosure.

Leg Elastics:

Leg elastic members, 166 and 168, can be secured between the outer and inner layers, 170 and 172, respectively, of the outer cover 126, such as by being bonded therebetween by laminate adhesive, generally adjacent the lateral outer edges of the inner layer 172 of the outer cover 126. Alternatively, the leg elastic members, 166 and 168, may be disposed between other layers of the absorbent article 110. A wide variety of elastic materials may be used for the leg elastic members, 166 and 168. Suitable elastic materials can include sheets, strands or ribbons of natural rubber, synthetic rubber, or thermoplastic elastomeric materials. The elastic materials can be stretched and secured to a substrate, secured to a gathered substrate, or secured to a substrate and then elasticized or shrunk, for example, with the application of heat, such that the elastic retractive forces are imparted to the substrate.

Fastening System:

In an embodiment, the absorbent article 110 can include a fastener system. The fastener system can include one or more back fasteners 240 and one or more front fasteners 242. Portions of the fastener system may be included in the front waist region 112, back waist region 114, or both. The fastener system can be configured to secure the absorbent article 110 about the waist of the wearer and maintain the absorbent article 110 in place during use. In an embodiment, the back fasteners 240 can include one or more materials bonded together to form a composite ear as is known in the art. For example, the composite fastener may be composed of a stretch component 244, a nonwoven carrier or hook base 246, and a fastening component 248.

Waist Elastic Members:

In an embodiment, the absorbent article 110 can have waist elastic members, 162 and 164, which can be formed of any suitable elastic material. In such an embodiment, suitable elastic materials can include, but are not limited to, sheets, strands or ribbons of natural rubber, synthetic rubber, or thermoplastic elastomeric polymers. The elastic materials can be stretched and bonded to a substrate, bonded to a gathered substrate, or bonded to a substrate and then elasticized or shrunk, for example, with the application of heat, such that elastic retractive forces are imparted to the substrate. It is to be understood, however, that the waist elastic members, 162 and 164, may be omitted from the absorbent article 110 without departing from the scope of this disclosure.

The absorbent structure 140 can be superposed over the inner layer 172 of the outer cover 126, extending laterally between the leg elastic members, 166 and 168, and can be bonded to the inner layer 172 of the outer cover 126, such as by being bonded thereto with adhesive. However, it is to be understood that the absorbent structure 140 may be in contact with, and not bonded with, the outer cover 126 and remain within the scope of this disclosure. In an embodiment, the outer cover 126 can be composed of a single layer and the absorbent body 140 can be in contact with the single layer of the outer cover 126. In an embodiment, a layer, such as but not limited to, a core wrap layer 178, can be positioned between the absorbent body 140 and the outer cover 126.

### EXAMPLES

### Example 1

To illustrate the ability of the closed cell foam matrix to contain and release oxygen, a number of samples were developed. The superabsorbent polymer material used in each of the samples was obtained from Evonik Stockhausen, LLC (Greensboro, N.C.) under the designation “SR1717” which is manufactured in accordance with U.S. Pat. No. 7,312,286. The superabsorbent material is an aqueous solution of sodium polyacrylate; as a 32% wt/wt solids in water solution.

A aqueous solution was prepared with 40 grams of the superabsorbent material (SR1717), 40 ml water, 10.5 ml 2N sodium hydroxide (a slight excess of base is added in order to neutralize the oligomeric acrylate that is present in the acid form), and 13.6 grams 17% hydrogen peroxide. The nonwoven and cellulose tissue described below in Table 1 were coated with the aqueous solution. The coating was applied via a dip & squeeze method using the Atlas wringer (Atlas Electric Devices, Chicago Ill. Model LWB24/LW-1), with 25 lb nip pressure. The samples were then dried in a convection oven (American Constant Temperature Oven Model DK-62, American Scientific Products) at 85° C. for 20 minutes.

The oxygen concentration in the coating was measured by taking a known weight of coated sample (˜100 mg) and placing it in 40 ml of nitrogen sparged (residual oxygen ˜0.8 ppm) deionized water for 5 minutes, while being held down on the bottom of the beaker using a spatula. The oxygen concentration in the water was then measured directly using a HACH dissolved oxygen (DO) probe, (Model HQ40d) from Ocean Optics, (Dunedin, Fla.) and is shown in Table 1.

TABLE 1

| | Aqueous | Aqueous | |
|---|---|---|---|
| | Solution | Solution | Concentration |
| | Dry | Dry | of Oxygen |
| | Add-on | Add-on | released |
| Sample | (g) | (%) | (ppm) |
| Thermal Bonded Airlaid | 0.04 | 2.6 | 35 |
| Spunlace | 0.09 | 16.0 | 80 |
| MFAL | 0.20 | 30.0 | 160 |
| Tissue wrap | 0.04 | 150 | 37 |
| Tissue wrap | 0.16 | 320 | 136 |

### Example 2

A aqueous solution was prepared with 40 grams of the superabsorbent material (SR1717), 40 ml water, 10.5 ml 2N sodium hydroxide (a slight excess of base is added in order to neutralize the oligomeric acrylate that is present in the acid form), and 13.6 grams 17% hydrogen peroxide. The sample was poured into a mold 4 mm thick 10.5×10.5 cm gel squares. The samples were then cut into four identical squares. Each was infused with an equivalent weight of 17% hydrogen peroxide. Once the material had absorbed all the peroxide liquid the sample was placed in a convection oven at 80° C. for 60-90 minutes to generate the foamed sample. Typically the sample doubles in size and thickness during the foam formation.

This sample was then broken up into chunks and placed in a coffee grinder (Smart Grind, model CBGS, Black & Decker, New Britain, Conn.) and processed to obtain white particles which were similar in size to sea salt.

Next, the powder was tested in nitrogen purged water to determine how much oxygen would be delivered by the powder. 0.12 g of powder was placed into 50 ml of nitrogen sparged water (1.8 ppm oxygen, 19.2° C.) and the oxygen released measured (HACH dissolved oxygen (DO) probe, model HQ40d) and found to be 15.2 ppm after 10 minutes and 14.1 ppm after 30 minutes. So it can be seen that converting the foam matrix into a powder does reduce the amount of oxygen delivered, however it is still enough to be a usable product in the powder form.

When introducing elements of the present disclosure or the preferred aspect(s) thereof, the articles “a,” “an,” and “the” are intended to mean that there are one or more of the elements. The terms “comprising,” “including,” and “having” are intended to be inclusive and mean that there can be additional elements other than the listed elements.

The disclosure has been described with reference to various specific and illustrative aspects and techniques. However, it should be understood that many variations and modifications can be made while remaining within the spirit and scope of the disclosure. Many alternatives, modifications and variations will be apparent to those skilled in the art in light of the foregoing description. Accordingly, this disclosure is intended to embrace all such alternatives, modifications, and variations that fall within the spirit and scope of the appended claims.

We claim:

1. An absorbent article that delivers oxygen to the skin, the absorbent article comprising:
a liquid permeable inner surface for facing the wearer;
an outer surface for facing away from the wearer;
an absorbent assembly disposed therebetween; and
a closed-cell foam matrix material applied to a portion of the absorbent article, the closed-cell foam matrix comprising:
a superabsorbent material comprising:
a. at least 15 percent by mass monoethylenically unsaturated carboxylic, sulphonic or phosphoric acid or salts thereof,
b. an acrylate or methacrylate ester that contains an alkoxysilane functionality,
c. a copolymerizable hydrophilic glycol containing ester monomer; and
oxygen entrapped within the closed-cell foam matrix material.

2. The absorbent article of claim 1 wherein the oxygen is produced by:
adding an alkali hydroxide catalyst and an oxygen precursor to an aqueous solution containing the superabsorbent material;
applying the portion of the absorbent article with the aqueous solution, and heating and foaming the coating to produce oxygen by reacting the alkali hydroxide catalyst and the oxygen precursor and to entrap the oxygen in the closed-cell foam matrix.

3. The absorbent article of claim 1 wherein monoethylenically unsaturated carboxylic, sulphonic or phosphoric acid or salts thereof comprises polyacrylic acid.

4. The absorbent article of claim 1 wherein the acrylate or methacrylate ester that contains an alkoxysilane functionality comprises methacryloxy-propyl-trimethoxylsilane.

5. The absorbent article of claim 1 wherein the copolymerizable hydrophilic glycol containing ester monomer comprises polyethylene glycol.

6. The absorbent article of claim 2 wherein the alkali hydroxide catalyst comprises sodium hydroxide.

7. The absorbent article of claim 2 wherein the alkali hydroxide catalyst is added at between about 0.5% to about 3% by weight of the liquid superabsorbent material.

8. The absorbent article of claim 2 wherein the oxygen precursor comprises hydrogen peroxide.

9. The absorbent article of claim 2 wherein the oxygen precursor is added at between about 15% to about 25% by weight of the liquid superabsorbent material.

10. The absorbent article of claim 1 wherein the closed-cell foam matrix material contains substantially no residual oxygen precursor or alkali hydroxide catalyst.

11. The absorbent article of claim 2 wherein a molar ratio of the alkali hydroxide catalyst to the oxygen precursor is in the range of 1.0:0.9 to 0.9:1.0 with the alkali hydroxide catalyst having an additional amount to neutralize the acid component superabsorbent material.

12. The absorbent article of claim 2 wherein the oxygen containing foamed matrix is in the form of a sheet, a coated or infused nonwoven matrix, fiber, or powder within the absorbent article.

13. The absorbent article of claim 1 wherein the superabsorbent material comprises an aqueous solution of an oligomeric polyacrylic acid having a silanol cross-linker covalently bonded to the backbone chain of a polyacrylic acid.

14. The absorbent article of claim 1 wherein the absorbent article is selected from a feminine pad, a diaper, an adult incontinence article, a training pant and a liner.

15. An method of forming a substrate that delivers oxygen, the method comprising:
adding an alkali hydroxide catalyst and an oxygen precursor to an aqueous solution containing a superabsorbent material, wherein the superabsorbent material comprises:
a. at least 15 percent by mass monoethylenically unsaturated carboxylic, sulphonic or phosphoric acid or salts thereof,
b. an acrylate or methacrylate ester that contains an alkoxysilane functionality,
c. a copolymerizable hydrophilic glycol containing ester monomer; and
applying the aqueous solution to a substrate; and
heating the substrate to produce oxygen by reacting the alkali hydroxide catalyst and the oxygen precursor and to entrap the oxygen in a closed-cell foam matrix.

16. The method of claim 15 wherein monoethylenically unsaturated carboxylic, sulphonic or phosphoric acid or salts thereof comprises polyacrylic acid.

17. The method of claim 15 wherein the acrylate or methacrylate ester that contains an alkoxysilane functionality comprises methacryloxy-propyl-trimethoxylsilane.

18. The method of claim 15 wherein the copolymerizable hydrophilic glycol containing ester monomer comprises polyethylene glycol.

19. The method of claim 15 wherein the alkali hydroxide catalyst comprises sodium hydroxide.

20. The method of claim 15 wherein the alkali hydroxide catalyst is added at between about 0. 5% to about 3% by weight of the liquid superabsorbent material.

21. The method of claim 15 wherein the oxygen precursor comprises hydrogen peroxide.

22. The method of claim 15 wherein the oxygen precursor is added at between about 15% to about 25% by weight of the liquid superabsorbent material.

23. The method of claim 15 wherein the oxygen precursor is the form of a sheet, coated or infused nonwoven matrix, fiber or powder.
